(19) United States
(12) Patent Application Publication
(10) Pub. No.: US 2024/0398886 A1
(43) Pub. Date: Dec. 5, 2024

(54) EFFECTIVE EYELID, FACE, HEAD AND BODY CLEANSER

(71) Applicant: Darren Stanley Morchesky, Punta Gorda, FL (US)

(72) Inventor: Darren Stanley Morchesky, Punta Gorda, FL (US)

(21) Appl. No.: 18/678,738

(22) Filed: May 30, 2024

Related U.S. Application Data
(60) Provisional application No. 63/505,136, filed on May 31, 2023.

(51) Int. Cl. A61K 36/58; A61K 9/00; A61K 31/35; A61K 47/18; A61K 47/26; A61K 47/44

(52) U.S. Cl. CPC A61K 36/58; A61K 9/0014; A61K 31/35; A61K 47/186; A61K 47/26; A61K 47/44

(57) ABSTRACT

Compositions for the treatment and removal of Demodex, Demodex blepharitis, debris and cosmetics from the eyelid, eyelashes, head, face and body are disclosed. The composition contains Neem infused water and/or Neem powder and/or Azadirachtin in solid form. The composition may further contain water or a mixture of glycerol and water, Cocamidopropyl Betaine, Decyl Glucoside, Organic Argan Oil, Phenoxyethanol, Ethylhexylglycerin, Polysorbate 80. All of the composition's ingredients may be mixed together, or packaged with the Neem powder and/or Azadirachtin in solid form, separated from other components. If packaged separately, the Neem powder and/or Azadirachtin in solid form can be distributed with the other ingredients simultaneously or nearly simultaneously, in such a way to be easily mixed together just prior to use. The composition may be mixed in hand or on any suitable brush, cloth, sponge, towel, bag, pouch or wipe, on desired areas of body, head, face and/or eyelids.

### CROSS-REFERENCE TO RELATED APPLICATIONS

This application claims the benefit of priority to U.S. Ser. No. 63/505,136, filed on May 31, 2023, the disclosure of which is incorporated herein by reference.

### FIELD OF THE INVENTION

The present invention relates to the composition and methods of treating Demodex infestation of eyelids, face, head and body resulting in Demodex blepharitis and Meibomian Gland Disorder with the use of Neem infused water and/or Neem powder and/or Azadirachtin in solid form, mixed in solution or with the Neem powder and/or Azadirachtin in solid form separated from the other ingredients, only to be mixed in solution at the time of use, on the eyelashes, eyelids, face, head and/or body.

### BACKGROUND OF THE INVENTION

Demodex, is a genus of tiny parasitic mites that live in hair follicles or sebaceous glands of mammals (FIG. 1). It's been reported that there are over 100 known species of Demodex, two of which, Demodex folliculorum and Demodex brevis, are found on humans. (18) Demodex mites belong to family Demodecidae, Subclass Arachnida and Phylum Arthropoda. Adult D. folliculorum mites can grow to 0.3-0.4 mm in length while the smaller D. brevis will grow to 0.15-0.2 mm in length. In darkness, these mites can move at a rate of 8-16 mm/h. In light, they prefer to recede into hair follicles or glands. The diet of Demodex consists of skin cells, hormones, and skin oils. They are most commonly found on the face, eye lashes, eye brows, scalp, neck, ears, but can also be found on other parts of the body. Demodex can transfer from one host to another through contact of hair, eyebrows, or sebaceous glands. Mating occurs in the hair follicle opening and eggs can be laid in either the hair follicles or the sebaceous glands. The larvae hatch after 3-4 days, and then develop into adults after 7 days. Demodex have a 14-day life cycle. After death, the mites decompose inside of hair follicles or sebaceous glands. (9) The decaying Demodex will combine with skin oils, skin cells and other debris to form eyelash collarettes, or what is more commonly known as Demodex Blepharitis. Demodex Blepharitis (FIG. 1) are tightly adhered to skin and hair follicles, and are not removed well by traditional facial cleansers or even modern eyelid cleansers. (FIG. 4, FIG. 5, FIG. 6, FIG. 7, FIG. 8)

Eyelids play an invaluable role in helping humans see. The eyelid dispenses meibum oil, produced in the Meibomian gland, to the tear film and distributes it evenly across the eye, resetting the protective tear film with every blink. The Meibomian gland is located next to the eyelashes. (FIG. 1) The outer tear film Lipid layer made of Meibum oil prevents evaporation of the middle aqueous tear film layer. The aqueous tear layer provides an extensive list of nutrients, enzymes and nerve growth factors to the cornea (FIG. 2) for the purpose of maintaining corneal health and clarity. Demodex Blepharitis, cosmetics, bacteria and other foreign material that adhere to eyelids and lashes, can initiate a change in the composition of the meibum oil that is stored in Meibomian glands. Without removal of this foreign matter, the meibum oil undergoes a progressive modification from a liquid into a solid. As the meibum oil hardens, it will not disperse well over the aqueous tear layer, allowing the nutrient rich tears to evaporate prematurely. This creates a break in the tear film and potentially a nutrient deficient cornea, which may result in conjunctivitis, infections, general discomfort, foreign body sensation, itching, blurry vision and even sight threatening conditions.

The Neem tree (Azadirachta indica), originates from India and has been used for centuries in medicine, skin therapies and insecticides. The Neem plant contains at least 100 biologically active compounds. The major elements are limonoids, the most important of those being azadirachtin. (FIG. 3) Other components include meliantriol, nimbin, nimbidin, nimbinin, nimbolides, salannin, tannins and various fatty acids (oleic, stearic, and palmitic). (10) Azadirachtin is considered non-toxic for humans and warm-blooded vertebrates and without genotoxicity for mammals. (12, 13) Azadirachtin affects most of the vital functions of arthropods and is a proven insecticide. It inhibits juvenile hormones that are essential for arthropod reproduction and development. Azadirachtin has anti-feedant and impaired development effects. It also induces an alteration of various reproductive processes, such as sterilization, fertility, oviposition, egg viability, oogenesis, vitellogenesis, spermiogenesis and gonad development. Azadirachtin also impacts the central nervous system by inhibiting excitatory cholinergic transmission via calcium channels. (11)

Current eyelid and facial cleansers do not effectively remove Demodex Blepharitis, eyelash debris and frequently leave microscopic cosmetic residue behind. Furthermore, these cleansers fail to provide a potent miticide to kill Demodex. Lingering foreign matter that accumulates on the eyelids affects the normal function of the eyelid that may result in conjunctivitis, infections, general discomfort, foreign body sensation, itching, blurry vision and even sight threatening conditions. Therefore, this invention is needed to restore proper eyelid capability and bring an end to the suffering of millions of people currently living with this chronic condition that decreases the quality of life and may even lead to vision loss.

### BRIEF SUMMARY OF THE INVENTION

Eyelash, eyelid, facial, head and body cleansing compositions made by infusing the properties of the Neem tree into water and methods of use thereof, are provided.

The compositions include Neem infused water at about 0.1-95% v/v, and/or Neem powder (about 0.1-95% w/v), and/or Azadirachtin in solid form (about 0.0001-10% w/v) in glycerol or in water or in a mixture of glycerol and water. The composition may further contain Cocamidopropyl Betaine (about 0.1-50% v/v), Decyl Glucoside (about 0.1-50% v/v), Organic Argan Oil (about 0.1-20% v/v), Kosher Vegetable Glycerin (about 0.1-20%, v/v), Phenoxyethanol (about 0.1-2%, v/v), Ethylhexylglycerin (about 0.1-5%, v/v), and Polysorbate (about 80, 0.1-10%, v/v).

The composition can be in the form of as a spray, lotion, shampoo, powder, liquid or solid. In some forms, the composition is incorporated into a body wash or shampoo.

The disclosed compositions are useful for the treatment of Demodex, Demodex blepharitis, and removal of dirt, debris and cosmetics. Neem infused water with the addition of other key ingredients effectively removes Demodex debris, including Demodex blepharitis, dirt and cosmetics from the body, head, face, eyelids and eyelashes. The disclosed compositions can have other uses such as insect repellants and treatment of insect infestation (lice, mites, scabies, bot flies, etc.)

Method of making the disclosed compositions are also provided. I, delaying introduction of Neem powder or Azadirachtin in solid form or a combination thereof to other ingredients just prior to use, prevents rapid degrading of anti-arthropod and miticidal properties that naturally occurs to them when placed into a solution. Therefore, these critical properties will be active against the Demodex mites. Additional function can be gained by mixing Neem infused water with key ingredients, to Neem powder and/or Azadirachtin in solid form, gaining the beneficial attributes of each into a single formula. Addition of the other ingredients help retain skin moisture and are in alignment with the gentle nature of the main Neem tree ingredients, allowing this to be used all over the body, head, face and eyelids.

### BRIEF DESCRIPTION OF THE DRAWINGS

FIG. 1. The human eyelid with Demodex Blepharitis coating the eyelashes, demodex mites inside eyelash follicles and Meibomian glands.

FIG. 2. Natural tear film and compromised tear film on human cornea.

FIG. 3. The Azadirachtin molecule has eight condensed rings, three carbocyclic and five heterocyclic, the latter of which contain oxygen as hetero atom which include one tetra-substituted oxirane, two five-membered, one tetrahydrofuran and one six membered pyran rings.

FIG. 4. Before picture shows makeup residue and Demodex Blepharitis from an individual who used a makeup remover and washed her face daily after last applying makeup 10 days prior. After picture shows eyelashes after only one cleansing with this invention for approximately 15 seconds with fingers.

FIG. 5. Before picture shows eyelids next day after using patented Neutrogena makeup removing wipe the night before. After picture shows eyelashes after only one cleansing with this invention for approximately 15 seconds with fingers.

FIG. 6. Before picture shows eyelids of individual using product with U.S. Pat. No. 10,172,902, for 3 years, with Demodex Blepharitis still prominent on the eyelid. After picture shows eyelashes after only one cleansing with this invention for approximately 15 seconds with fingers.

FIG. 7. Before picture shows eyelids of individual with severe Demodex Blepharitis, with symptoms of fluctuating vision, itchiness, eye pain. After picture shows eyelashes after only one cleansing with this invention for approximately 25 seconds with fingers.

FIG. 8. Before picture shows makeup residue and Demodex Blepharitis from an individual who used a makeup remover and washed her face after last applying makeup the previous day. After picture shows eyelashes after only one cleansing with this invention for approximately 15 seconds with fingers.

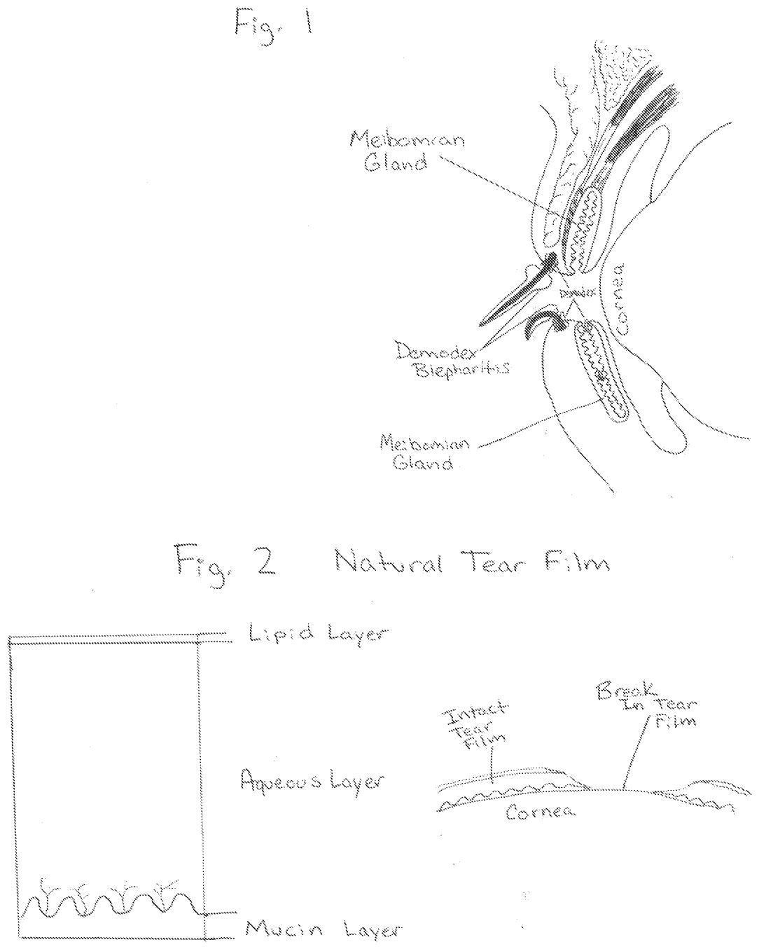

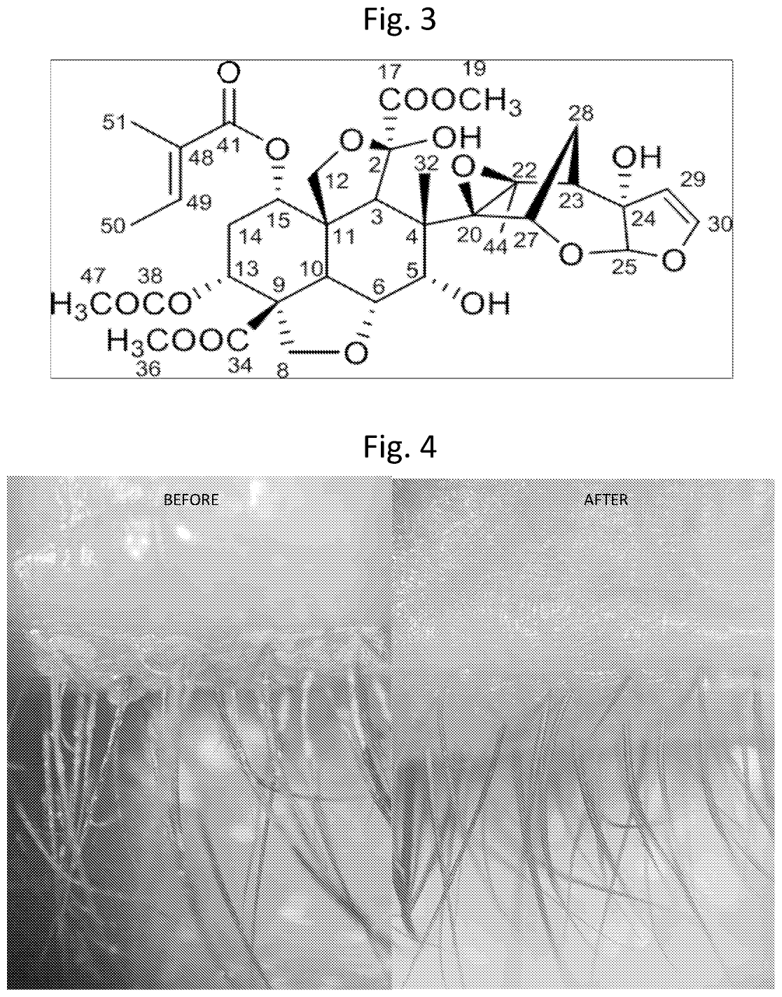

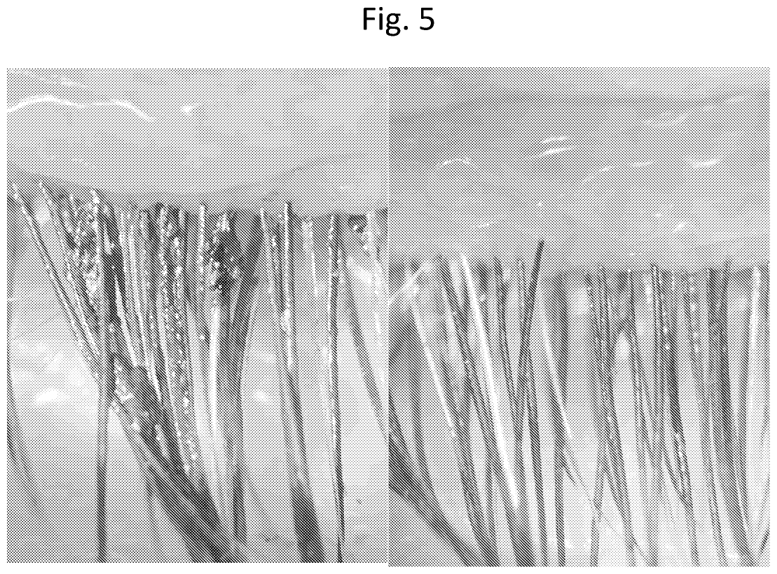

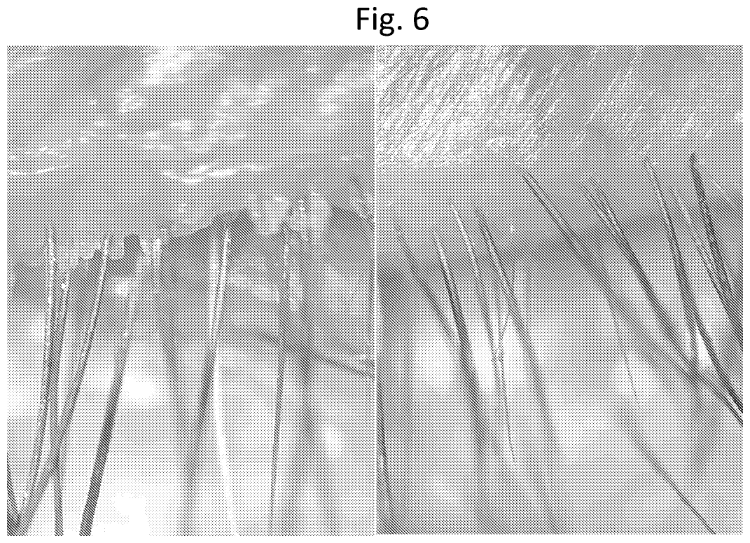

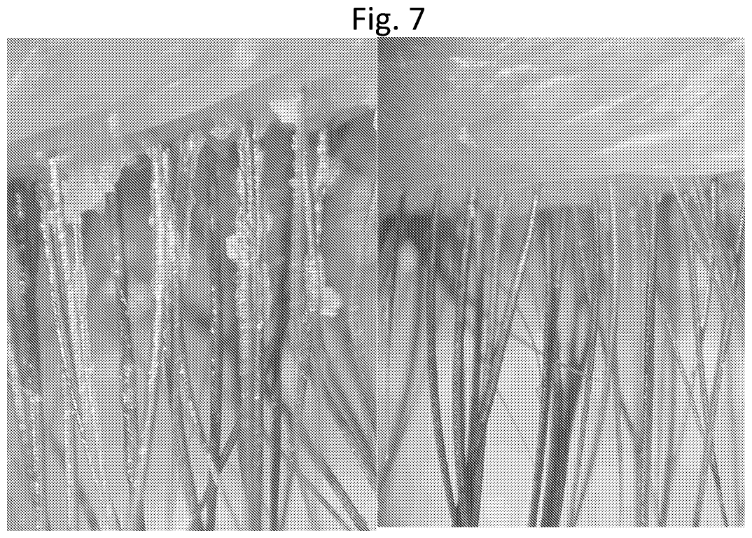

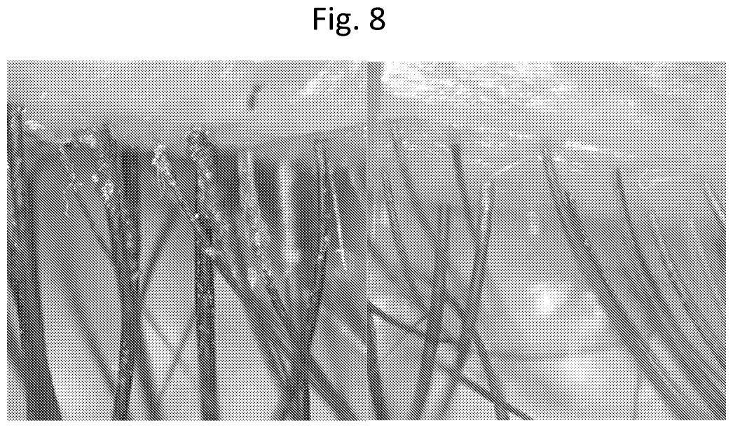

### DETAILED DESCRIPTION OF THE INVENTION

Although the description provided below outlines the preferred embodiments of the present invention, it should be noted that the invention is not restricted to the specifics of the disclosed information and descriptions in the accompanying specifications. The invention has the potential to take on alternative forms and can be utilized in different ways.

### I. Definitions

A “cream” is a viscous liquid or semi-solid emulsion of either the “oil-in-water” or “water-in-oil type”.

An “emulsion” is a composition containing a mixture of non-miscible components homogenously blended together.

“Gel” as used herein is a colloid in which the dispersed phase has combined with the continuous phase to produce a semisolid material, such as jelly.

A “lotion” is a low- to medium-viscosity liquid formulation.

A “subject” or “patient” refers to a human, primate, non-human primate, laboratory animal, farm animal, livestock, or a domestic pet.

“Therapeutically effective” or “effective amount” as used herein means that the amount of the composition used is of sufficient quantity to ameliorate one or more causes or symptoms of a disease or disorder. Such amelioration only requires a reduction or alteration, not necessarily elimination. As used herein, the terms “therapeutically effective amount” “therapeutic amount” and “pharmaceutically effective amount” are synonymous. One of skill in the art can readily determine the proper therapeutic amount.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

### II. Compositions

In some forms, Neem infused water, is formulated at about 0.1-95% w/v, preferably about 0.6% w/v, to be used in the following formulation: Neem infused water is about 0.1-95% v/v, preferably about 53%, v/v, Neem powder about 0.1-95% w/v, preferably about 1%, w/v, Azadirachtin in solid form about 0.0001-10% w/v, preferably about 0.1% w/v in substances such as glycerol or in water or in a mixture of glycerol and water. Other ingredients that can be combined with Neem infused water and/or Neem powder and/or Azadirachtin in solid form are Cocamidopropyl Betaine about 0.1-50% v/v, preferably about 15%, v/v, Decyl Glucoside about 0.1-50% v/v, preferably about 10%, v/v, Organic Argan Oil about 0.1-20% v/v, preferably about 10%, v/v, Kosher Vegetable Glycerin about 0.1-20%, v/v, preferably about 10%, v/v, Phenoxyethanol about 0.1-2%, v/v, preferably about 1%, v/v, Ethylhexylglycerin about 0.1-5%, v/v, preferably about 0.5%, v/v, Polysorbate 80, about 0.1-10% v/v, preferably about 0.5%, v/v.

In some forms, an advantage of the disclosed compositions is the water-based solution that contains Neem properties to effectively strip away unwanted Demodex based deposits from skin and hair. Another advantage of the disclosed compositions is the water-based solution which dissolves the highly water soluble Azadirachtin in solid form, immediately on contact. Another advantage of the disclosed compositions is the combination of glycerol to the Neem ingredients, which will help retain the skins moisture as the unwanted Demodex material is removed from the skin and hair.

Another advantage of the disclosed compositions is combining the debris removing ability of Neem Infused water with the potent miticide activity of Neem powder and/or Azadirachtin in solid form when mixed together at time of use.

Another advantage of this invention is the gentle nature of the cleanser, which allows it to be used to treat Demodex on all areas of body, head, face and eyelids, removing them from your eyelids and preventing other demodex mites from migrating to your eyes.

The disclosed compositions are preferably formulated for topical administration.

Suitable dosage forms for topical administration include creams, ointments, salves, sprays, gels, lotions, emulsions, and transdermal patches. The compositions may further contain one or more chemical penetration enhancers, membrane permeability agents, membrane transport agents, emollients, surfactants, stabilizers, buffers, and combination thereof.

In certain embodiments, it may be desirable to provide continuous delivery of one or more compounds to a patient in need thereof. For topical applications, repeated application can be done or a patch can be used to provide continuous administration of the compounds over an extended period of time

“Buffers” are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

“Emollients” are an externally applied agent that softens or soothes skin and are generally known in the art and listed in compendia, such as the “Handbook of Pharmaceutical Excipients”, 4th Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

“Penetration enhancers” are known in the art and include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols). Examples of these classes are known in the art.

“Preservatives” can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

### (a). Emulsions

An emulsion is a preparation of one liquid distributed in small globules throughout the body of a second liquid. In particular embodiments, the non-miscible components of the emulsion include a lipophilic component and an aqueous component. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These drug delivery systems are typically capsules (hard shell or soft shell) comprised of the drug dispersed or dissolved in a mixture of surfactant(s) and lipophilic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

### (b). Lotions

A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

### (c). Creams

Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments, as they are generally easier to spread and easier to remove.

The difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

### (d). Ointments

Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

### (e). Gels

Gels are semisolid systems containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

### (f). Foams

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases, which can produce flammable or explosive vapors during spraying. Furthermore, the compositions preferably contain no volatile alcohols, which can produce flammable or explosive vapors during use.

### III. Methods of Making

All of the composition's ingredients may be mixed together, or packaged with the Neem powder and/or Azadirachtin in solid form, separated from the other components. If packaged separately, the Neem powder and/or Azadirachtin in solid form can be dispensed with the other ingredients simultaneously or nearly simultaneously, in such a way to be easily mixed together just prior to use. The composition may be mixed in hand or on any suitable brush, cloth, sponge, towel, bag, pouch or wipe. Once the premixed solution and/or the Neem powder and/or Azadirachtin in solid form is dispensed and mixed with the other ingredients, it is then applied on the eyelashes, eyelids, face, head and/or body to treat Demodex infestation, Demodex blepharitis and to remove other dirt, debris and cosmetics.

In some forms, an advantage of the disclosed methods of formulating the disclosed compositions is delaying the rapid degradation of the biologically active compounds by withholding the mixture of Neem powder and/or Azadirachtin in solid form, from water and/or solution until the time of use, which provides a potent miticide to the affected areas.

Neem powder is made from the leaves, bark, root or fruit of the Neem tree. The chosen material is dried and ground into a powder. Neem powder is kept in an airtight container, that protects it from water, humidity and light to prevent the important elements with miticidal activity from degrading, until use.

Neem infused water production starts with water that has been treated using industry standard processes to ensure it is suitable for cosmetic ingredient production and can be laboratory verified as pure H20. The process involves treating the water multiple times to demineralize, filter and disinfect it. Neem tree material, ranging in size from large pieces of bark, fruit, roots or whole leaves to finely ground powder, is added to a filtering device with openings smaller than the size of the smallest material used. Due to the potentially wide range in size of Neem Tree material, these filter openings can range in size from as small as 1 micron to as large as 3 cm. The filter device is then placed inside a larger container with water that has been heated to 60-100 degrees. Our concentration of Neem tree material in the water is 0.1-95% w/v, preferably 0.6% w/v. The Neem material is left in the water for up to three days before the filter containing the Neem material is removed to obtain the desired concentration of Neem infused water. The Neem infused water can then be combined with other ingredients for use. The preferred method includes using water initially heated to 65 degrees Celsius with finely ground Neem powder, then allowing the water to naturally cool to room temperature with Neem material in water for 1 day. It should be noted that larger Neem material, other water temperatures and duration of Neem material in water can be used to make Neem infused water.

### IV. Methods of Using

The disclosed compositions are useful for the treatment of Demodex, Demodex blepharitis, and removal of dirt, debris and cosmetics. Neem infused water with the addition of other key ingredients effectively removes Demodex debris, including Demodex blepharitis, dirt and cosmetics from the body, head, face, eyelids and eyelashes. The disclosed compositions can have other uses such as insect repellants and treatment of insect infestation (lice, mites, scabies, bot flies, etc.)

### V. Kits

All of the composition's ingredients may be mixed together, or packaged with the Neem powder and/or Azadirachtin in solid form, separated from other components. If packaged separately, the Neem powder and/or Azadirachtin in solid form can be distributed with the other ingredients simultaneously or nearly simultaneously, in such a way to be easily mixed together just prior to use.

### EXAMPLES

### Example 1

Neem infused water, is formulated at 0.6% w/v, to be used in the following formulation: Neem infused water 53%, v/v, Cocamidopropyl Betaine 15%, v/v, Decyl Glucoside 10%, v/v, Organic Argan Oil 10%, v/v, Kosher Vegetable Glycerin 10%, v/v, Phenoxyethanol 1%, v/v, Ethylhexylglycerin 0.5%, v/v, Polysorbate 80, 0.5%, v/v.

### Example 2

Neem powder 1%, w/v, is isolated in a dry filter bag. The filter bag is moistened by a solution containing Cocamidopropyl Betaine 0.1-50% v/v, Decyl Glucoside 0.1-50% v/v, Organic Argan Oil 0.1-20% v/v, Kosher Vegetable Glycerin 0.1-20%, v/v, Phenoxyethanol 0.1-2%, v/v, Ethylhexylglycerin 0.1-5%, v/v, Polysorbate 80, 0.1-10%, v/v, or a combination thereof, in glycerol or in water or in any desired mixture of glycerol and water. After ensuring the bags contents are sufficiently wetted by the solution, it is used to gently scrub desired areas of eyelashes, eyelids, body, head and/or face.

### Example 3

Azadirachtin in solid form 0.1% w/v, is embedded into a disposable wipe. The disposable wipe is moistened by a solution containing Cocamidopropyl Betaine 0.1-50% v/v, Decyl Glucoside 0.1-50% v/v, Organic Argan Oil 0.1-20% v/v, Kosher Vegetable Glycerin 0.1-20%, v/v, Phenoxyethanol 0.1-2%, v/v, Ethylhexylglycerin 0.1-5%, v/v, Polysorbate 80, 0.1-10%, v/v, or a combination thereof, in glycerol or in water or in any desired mixture of glycerol and water. After ensuring the wipe contents are sufficiently wetted by the solution, it is used to gently scrub desired areas of eyelashes, eyelids, body, head and/or face.

### Example 4

Azadirachtin in solid form 2.1% w/v, is contained by a water dissolvable film, held securely inside of a sponge, pouch or bag. The sponge, pouch or bag may or may not have other elements embedded in it. The sponge, pouch or bag may be moistened with water or moistened by solution containing Cocamidopropyl Betaine 0.1-50% v/v, Decyl Glucoside 0.1-50% v/v, Organic Argan Oil 0.1-20% v/v, Kosher Vegetable Glycerin 0.1-20%, v/v, Phenoxyethanol 0.1-2%, v/v, Ethylhexylglycerin 0.1-5%, v/v, Polysorbate 80, 0.1-10%, v/v, or a combination thereof, in glycerol or in water or in any desired mixture of glycerol and water. After ensuring the wipe contents are sufficiently wetted by the solution, it is used to gently scrub desired areas of eyelashes, eyelids, body, head and/or face.

### Example 5

Azadirachtin in solid form 0.6% w/v, is contained by a water dissolvable film, held securely inside of a container. The container holds multiple units of single use of Azadirachtin in solid form contained inside a water dissolvable film, and allows for singular distribution of these units. Once distributed, these single use units can then further be mixed with water or moistened by solution containing Cocamidopropyl Betaine 0.1-50% v/v, Decyl Glucoside 0.1-50% v/v, Organic Argan Oil 0.1-20% v/v, Kosher Vegetable Glycerin 0.1-20%, v/v, Phenoxyethanol 0.1-2%, v/v, Ethylhexylglycerin 0.1-5%, v/v, Polysorbate 80, 0.1-10%, v/v, or a combination thereof, in glycerol or in water or in any desired mixture of glycerol and water. After ensuring the Azadirachtin in solid form is sufficiently mixed with the water and/or solution, it is used with any suitable brush, cloth, sponge, towel, bag, pouch, wipe or clean fingers to gently scrub desired areas of eyelashes, eyelids, body, head and/or face.

### Example 6

Azadirachtin in solid form 0.2% w/v, is formed into a ball, cube, triangle, cylinder, disc or other shape, that allows it to contain the correct amount of Azadirachtin for single use and for it to be distributed as a single use unit. These formed shapes of Azadirachtin can be held in a multiple use container, or individually wrapped. Once distributed, these single use units can then further be mixed with water or moistened by solution containing Cocamidopropyl Betaine 0.1-50% v/v, Decyl Glucoside 0.1-50% v/v, Organic Argan Oil 0.1-20% v/v, Kosher Vegetable Glycerin 0.1-20%, v/v, Phenoxyethanol 0.1-2%, v/v, Ethylhexylglycerin 0.1-5%, v/v, Polysorbate 80, 0.1-10%, v/v, or a combination thereof, in glycerol or in water or in any desired mixture of glycerol and water. After ensuring the Azadirachtin in solid form is sufficiently mixed with the water and/or solution, it is used with any suitable brush, cloth, sponge, towel, bag, pouch, wipe or clean fingers to gently scrub desired areas of eyelashes, eyelids, body, head and/or face.

### Example 7

Azadirachtin in solid form 1.5% w/v, is held in a container, that is adjacent to another container holding a solution of Cocamidopropyl Betaine 0.1-50% v/v, Decyl Glucoside 0.1-50% v/v, Organic Argan Oil 0.1-20% v/v, Kosher Vegetable Glycerin 0.1-20%, v/v, Phenoxyethanol 0.1-2%, v/v, Ethylhexylglycerin 0.1-5%, v/v, Polysorbate 80, 0.1-10%, v/v, or a combination thereof, in glycerol or in water or in any desired mixture of glycerol and water. The 2 containers are packaged side by side with 1 single pump that simultaneously draws from each container, dispensing the contents onto a hand, suitable brush, cloth, sponge, towel, bag, pouch or wipe where it can be mixed and then used to gently scrub desired areas of eyelashes, eyelids, body, head and/or face.

### Example 8

Neem infused water, is formulated at 10% w/v, for use in the following formulation. Neem powder 20%, w/v, is isolated in an individually wrapped dry filter bag. The filter bag is moistened by a solution of Neem infused water 35%, v/v, Cocamidopropyl Betaine 0.1-50% v/v, Decyl Glucoside 0.1-50% v/v, Organic Argan Oil 0.1-20% v/v, Kosher Vegetable Glycerin 0.1-20%, v/v, Phenoxyethanol 0.1-2%, v/v, Ethylhexylglycerin 0.1-5%, v/v, Polysorbate 80, 0.1-10%, v/v, or a combination thereof, in glycerol or in water or in any desired mixture of glycerol and water. After ensuring the bags contents are sufficiently wetted by the solution, it is used to gently scrub desired areas of eyelashes, eyelids, body, head and/or face.

### Example 9

Neem infused water, is formulated at 1.2% w/v, to be used in the following formulation: Neem infused water 45%, v/v, Azadirachtin in solid form 10%, w/v, Cocamidopropyl Betaine 21%, v/v, Organic Argan Oil 12%, v/v, Decyl Glucoside 10%, v/v, Kosher Vegetable Glycerin 10%, v/v, Phenoxyethanol 1%, v/v, Ethylhexylglycerin 0.5%, v/v, Polysorbate 80, 0.5%, v/v. The solution is then used to gently scrub desired areas of eyelashes, eyelids, body, head and/or face.

### Example 10

Neem powder 15%, w/v, and Azadirachtin in solid form 0.003%, w/v, are isolated in a dry filter bag. The filter bag is moistened by a solution of Cocamidopropyl Betaine 0.1-50% v/v, Decyl Glucoside 0.1-50% v/v, Organic Argan Oil 0.1-20% v/v, Kosher Vegetable Glycerin 0.1-20%, v/v, Phenoxyethanol 0.1-2%, v/v, Ethylhexylglycerin 0.1-5%, v/v, Polysorbate 80, 0.1-10%, v/v, or a combination thereof, in glycerol or in water or in any desired mixture of glycerol and water. After ensuring the bags contents are sufficiently wetted by the water or solution, it is used to gently scrub desired areas of eyelashes, eyelids, body, head and/or face.

### Example 11

Neem infused water, is formulated at 0.8% w/v, to be used in the following formulation: Neem powder 25%, w/v, and Azadirachtin in solid form 0.05%, w/v, are isolated in a dry filter bag. The filter bag is moistened by a solution of Neem infused water 0.1-95% v/v, Cocamidopropyl Betaine 0.1-50% v/v, Decyl Glucoside 0.1-50% v/v, Organic Argan Oil 0.1-20% v/v, Kosher Vegetable Glycerin 0.1-20%, v/v, Phenoxyethanol 0.1-2%, v/v, Ethylhexylglycerin 0.1-5%, v/v, Polysorbate 80, 0.1-10%, v/v, or a combination thereof, in glycerol or in water or in any desired mixture of glycerol and water. After ensuring the bags contents are sufficiently wetted by the solution, it is used to gently scrub desired areas of eyelashes, eyelids, body, head and/or face.

### Example 12

Azadirachtin in solid form 3.8% w/v, is individually wrapped and held inside a pouch that prior to use can be moistened with water or moistened by a solution of Cocamidopropyl Betaine 0.1-50% v/v, Decyl Glucoside 0.1-50% v/v, Organic Argan Oil 0.1-20% v/v, Kosher Vegetable Glycerin 0.1-20%, v/v, Phenoxyethanol 0.1-2%, v/v, Ethylhexylglycerin 0.1-5%, v/v, Polysorbate 80, 0.1-10%, v/v, or a combination thereof, in glycerol or in water or in any desired mixture of glycerol and water. After ensuring the pouches contents are wetted by water or the solution, it is used to gently scrub desired areas of on the eyelashes, eyelids, face, head and/or body.

### Example 13

Azadirachtin in solid form 0.8% w/v, is contained inside of a device, that is able to store multiple, single use servings of Azadirachtin in solid form, and dispense the appropriate amount of Azadirachtin for a single use onto a hand, suitable brush, cloth, sponge, towel, bag, pouch or wipe. The chosen scrubbing medium may be fixed to the dispensing device or able to be disconnected so new medium could be attached or not attached or connected to device. This may be moistened with water or moistened by a solution of Cocamidopropyl Betaine 0.1-50% v/v, Decyl Glucoside 0.1-50% v/v, Organic Argan Oil 0.1-20% v/v, Kosher Vegetable Glycerin 0.1-20%, v/v, Phenoxyethanol 0.1-2%, v/v, Ethylhexylglycerin 0.1-5%, v/v, Polysorbate 80, 0.1-10%, v/v, or a combination thereof, in glycerol or in water or in any desired mixture of glycerol and water. After mixing of Azadirachtin in solid form with the wetting agent, the chosen medium is used to gently scrub the desired areas of the eyelashes, eyelids, face, head and/or body.

### Example 14

Azadirachtin in solid form 0.4% w/v, and/or Neem powder 3.0% w/v is contained inside of a glove. The glove may be moistened with water or moistened by a solution of Cocamidopropyl Betaine 0.1-50% v/v, Decyl Glucoside 0.1-50% v/v, Organic Argan Oil 0.1-20% v/v, Kosher Vegetable Glycerin 0.1-20%, v/v, Phenoxyethanol 0.1-2%, v/v, Ethylhexylglycerin 0.1-5%, v/v, Polysorbate 80, 0.1-10%, v/v, or a combination thereof, in glycerol or in water or in any desired mixture of glycerol and water. After mixing the glove containing Azadirachtin in solid form 0.4% w/v, and/or Neem powder 3.0% w/v with the wetting agent, the chosen medium is used to gently scrub desired areas of the eyelashes, eyelids, face, head and/or body.

### Example 15

Azadirachtin in solid form 0.25% w/v, and/or Neem powder 4.0% w/v is contained inside of a finger sleeve. The finger sleeve may cover one or more fingers. The finger sleeve may be moistened with water or moistened by a solution of Cocamidopropyl Betaine 0.1-50% v/v, Decyl Glucoside 0.1-50% v/v, Organic Argan Oil 0.1-20% v/v, Kosher Vegetable Glycerin 0.1-20%, v/v, Phenoxyethanol 0.1-2%, v/v, Ethylhexylglycerin 0.1-5%, v/v, Polysorbate 80, 0.1-10%, v/v, or a combination thereof, in glycerol or in water or in any desired mixture of glycerol and water. After mixing the finger sleeve containing Azadirachtin in solid form 0.25% w/v, and/or Neem powder 4.0% w/v with the wetting agent, the chosen medium is used to gently scrub desired areas of the eyelashes, eyelids, face, head and/or body.

### Example 16

A person who has Demodex-induced blepharitis in the eyelid can be provided with a composition that contains Neem infused water and/or Neem powder and/or Azadirachtin in solid form, as active agents. The composition should be applied to the eyelid margin and eyelashes. To cleanse the eyelid margin and eyelashes, the composition should be gently scrubbed using any suitable brush, cloth, sponge, towel, bag, pouch, wipe or clean fingers.

### Example 17

A person who has a Demodex scalp infestation can be provided with a composition that contains Neem infused water and/or Neem powder and/or Azadirachtin in solid form, as active agents. The composition should be applied to the scalp and hair. To cleanse the scalp and hair, the composition should be gently scrubbed using any suitable brush, cloth, sponge, towel, bag, pouch, wipe or clean fingers.

### Example 18

A person who has a Demodex-induced blepharitis, and Demodex scalp infestation can be provided with a composition that contains Neem infused water and/or Neem powder and/or Azadirachtin in solid form, as active agents. The composition should be applied to the eyelid margin, eyelashes, eyebrows, ears, face, scalp, hair and/or body and gently scrubbed using any suitable brush, cloth, sponge, towel, bag, pouch, wipe or clean fingers.

Although specific exemplary embodiments have been described in some detail, it is not the intention to limit the invention to such detail. Rather, the invention is intended to encompass alternative or equivalent embodiments that are consistent with the spirit and scope of the invention, as set forth and claimed herein.

### REFERENCES

1 Rather P A, Hassan I. Human demodex mite: the versatile mite of dermatological importance. Indian J Dermatol. 2014 January; 59 (1): 60-6. doi: 10.4103/0019-5154.123498.
2. Campos Estefãnia V. R., de Oliveira Jhones L., Pascoli Mônica, de Lima Renata, Fraceto Leonardo F. (2016) Neem Oil and Crop Protection: From Now to the Future. Frontiers in Plant Science, 7:1435. doi: 10.3389/fpls.2016.01435.
3. Aribi N, Denis B, Kilani-Morakchi S, Joly D. L'azadirachtine, un pesticide naturel aux effets multiples. Med Sci (Paris). 2020 January; 36 (1): 44-49. doi: 10.1051/medsci/2019247.
4. Braga T M, Rocha L, Chung T Y, Oliveira R F, Pinho C, Oliveira A I, Morgado J, Cruz A. Azadirachta indica A. Juss. In Vivo Toxicity—An Updated Review. Molecules. 2021 Jan. 6; 26 (2): 252. doi: 10.3390/molecules26020252. PMID: 33419112; PMCID: PMC7825405.
5. United States Environmental Protection Agency. (1996). RED FACTS—Neem Oil.
6. Aktaş Karabay E, Aksu Çerman A. Demodex folliculorum infestations in common facial dermatoses: acne vulgaris, rosacea, seborrheic dermatitis. An Bras Dermatol. 2020 March-April; 95 (2): 187-193. doi: 10.1016/j.abd.2019.08.023. Epub 2020 Feb. 12. PMID: 32113677; PMCID: PMC7175027.
7 Maldonado-Gómez W, Guevara-Sánchez E, Guevara-Vásquez G, Mera-Villasis K, Munayco C V. Association Between Demodex Infestation and Severe Acne Vulgaris: A Cross-Sectional Study of 168 Patients. Actas Dermosifiliogr. 2022 September; 113 (8): 758-764. English, Spanish. doi: 10.1016/j.ad.2022.03.011. Epub 2022 Apr. 30. PMID: 35504311.
8. Jarmuda S, O'Reilly N, Żaba R, Jakubowicz O, Szkaradkiewicz A, Kavanagh K. Potential role of Demodex mites and bacteria in the induction of rosacea. J Med Microbiol. 2012 November; 61 (Pt 11): 1504-1510. doi: 10.1099/jmm.0.048090-0. Epub 2012 Aug. 29. PMID: 22933353.
9. N. Lacey and others, Mite-related bacterial antigens stimulate inflammatory cells in rosacea, British Journal of Dermatology, Volume 157, Issue 3, 1 Sep. 2007, Pages 474-481,
10. Lacey N, Kavanagh K, Tseng S C. Under the lash: Demodex mites in human diseases. Biochem (Lond). 2009 Aug. 1; 31 (4): 2-6. PMID: 20664811; PMCID: PMC2906820.

Publications cited herein and the material for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

I claim:

1. A composition for the treatment of Demodex, Demodex blepharitis, and removal of dirt, debris and cosmetics comprising: Neem Infused water and Cocamidopropyl Betaine in glycerol or in water or in a mixture of glycerol and water.

2. The composition of claim 1 further comprising Decyl Glucoside.

3. The composition of claim 1 further comprising Organic Argan Oil.

4. The composition of claim 1 further comprising Neem powder already included or combined just prior to consumer use.

5. The composition of claim 1 further comprising Azadirachtin in solid form already included or combined just prior to consumer use.

6. A composition for the treatment of Demodex, Demodex blepharitis, and removal of dirt, debris and cosmetics comprising: Neem powder already combined with the following ingredients or combined just prior to consumer use with Cocamidopropyl Betaine in glycerol or in water or in a mixture of glycerol and water

7. The composition of claim 6 further comprising Organic Argan Oil.

8. The composition of claim 6 further comprising Decyl Glucoside.

9. The composition of claim 6 further comprising Azadirachtin in solid form already included or combined just prior to consumer use.

10. A composition for the treatment of Demodex, Demodex blepharitis, and removal of dirt, debris and cosmetics comprising: Azadirachtin in solid form already combined with the following ingredients or combined just prior to consumer use with glycerol or in water or in a mixture of glycerol and water.

11. The composition of claim 10 further comprising Organic Argan Oil.

12. The composition of claim 10 further comprising Decyl Glucoside.

13. The composition of claim 10 further comprising Neem Infused water.

14. The composition of claim 10 further comprising Cocamidopropyl Betaine.

15. A method of treating a subject in need thereof, comprising administering the composition of claim 1 on the subject.

16. The method of claim 15, wherein the subject is human subject.

17. The method of claim 15, comprising administering the composition to the eyelids, eyelashes, head, face or body of the subject.

18. The method of claim 17, wherein the composition is applied to the head in the firm of a shampoo.

19. The method of claim 17, wherein the composition is applied to the body in the form of a body wash.

20. The method of claim 17, wherein the composition is applied to the body in the form of a spray as an insect repellant.
